(19) United States
(12) Patent Application Publication
(10) Pub. No.: US 2008/0295206 A1
(43) Pub. Date: Nov. 27, 2008

(54) P450 Polynucleotides, Polypeptides, and Uses Thereof

(75) Inventor: Zhihong C. Cook, Laguna Hills, CA (US)

Correspondence Address: FISH & RICHARDSON P.C., P.O. BOX 1022, MINNEAPOLIS, MN, 55440-1022, US

(73) Assignee: Ceres, Inc., Thousand Oaks, CA (US)

(21) Appl. No.: 11/864,470

(22) Filed: Sep. 28, 2007

Related U.S. Application Data
(62) Division of application No. 11/208,308, filed on Aug. 19, 2005, now abandoned.
(60) Provisional application No. 60/603,533, filed on Aug. 20, 2004.

(51) Int. Cl. C12N 15/11

(52) U.S. Cl. 800/287; 800/290

(57) ABSTRACT

Isolated P₄₅₀ polynucleotides and polypeptides are disclosed, including isolated cpd polynucleotide and CPD polypeptide sequences. The polypeptides can be orthologous CPD polypeptides to Arabidopsis CPD. Recombinant vectors, host cells, transgenic plants, and seeds that include the polynucleotides and/or polypeptides are also disclosed, as well as methods for preparing and using the same.

### CROSS-REFERENCE TO RELATED APPLICATIONS

This application is a divisional application that claims priority to U.S. Provisional Application Ser. No. 60/603,533, filed on Aug. 20, 2004, and U.S. Utility application Ser. No. 11/208,308, filed on Aug. 19, 2005, incorporated by reference in their entirety herein.

### TECHNICAL FIELD

This invention relates to polynucleotides that encode polypeptides, including polypeptides that function in the brassinosteroid biosynthesis pathway, and more particularly to polynucleotides encoding cytochrome P₄₅₀ polypeptides, transgenic plants and plant cells including the same, and methods for modifying plant characteristics using the same.

### BACKGROUND

Increased demands on the agricultural and forestry industries due to world-wide population growth have resulted in efforts to increase plant production and/or size. Although one means for increasing plant size is through plant breeding programs, such breeding programs are typically time-consuming and labor-intensive. Genetic manipulation of plant characteristics through the introduction of exogenous nucleic acids conferring a desirable trait, on the other hand, can be less time-consuming and possibly applicable across a variety of plant species.

Plants produce a number of steroids and sterols, termed brassinosteroids (BRs), some of which function as growth-promoting hormones. There are over 40 BRs known, typically with characteristic oxygen moieties at one or more of the C-2, C-6, C-22, and C-23 positions. Brassinolide (BL) is the most bioactive form of the growth-promoting BRs. Arabidopsis CPD and DWF4 are cytochrome P₄₅₀ proteins that catalyze enzymatic steps in the BL biosynthetic pathway; they are 43% identical at the amino acid level. During the biosynthesis of BL, DWF4 catalyzes the oxidation of campestanol at C-22 to form 6-deoxocathasterone, while CPD catalyzes the adjacent step downstream, the hydroxylation of 6-deoxocathasterone at C-23 to produce 6-deoxoteasterone.

### SUMMARY

Provided herein are orthologous polypeptides to the Arabidopsis P₄₅₀ protein known as CPD (SEQ ID NO:2) and isolated polynucleotides that encode such polypeptides; transgenic plants and plant cells that include such polynucleotides; seeds, food products, animal feed, and articles of manufacture derived from transgenic plants; and methods employing the same. CPD plays an important role in the synthesis of brassinosteroids, which function as plant growth-promoting hormones. Such CPD polypeptides can function in the brassinosteroid biosynthesis pathway. For example, some of the polypeptides can perform the enzymatic activity of CPD, e.g., hydroxylation of 6-deoxocathasterone at C-23 to produce 6-deoxoteasterone. Expression of the polypeptides in plants can result in phenotypic effects, such as increased plant size (e.g., height) and/or a more rapid rate of growth. In other cases, expression of the polypeptides can provide biochemical or enzymatic activities not normally present in the plant (e.g., not present at all or only in certain tissues). In certain cases, expression of the polypeptides can complement biochemical or enzymatic functions already present in the plant, or can result in altered enzymatic activity (e.g., increased activity, decreased activity, or a different activity). Inhibition of expression of such CPD polypeptides in plants, e.g., by antisense, RNAi, or ribozyme-based methods, can result in improved shade tolerance of the plants.

Accordingly, in one embodiment, an isolated polynucleotide comprising a nucleic acid encoding a polypeptide having:
(a) about 80% or greater sequence identity to the GmCPD1 amino acid sequence set forth in SEQ ID NO:8
(b) about 90% or greater sequence identity to each of domain A, domain B, and the heme-binding domain of GmCPD1; and
(c) about 80% or greater sequence identity to domain C of GmCPD1 is provided. The polypeptide can be effective for catalyzing the hydroxylation of 6-deoxocathasterone at C-23 to produce 6-deoxoteasterone. An Arabidopsis plant, when expressing the polypeptide, can exhibit a height at least about 7% greater than an Arabidopsis plant not expressing said polypeptide. Expression can be under the control of a tissue specific promoter and can be measured in T3 Arabidopsis plants using RT-PCR. A polypeptide can have greater than about 85% sequence identity, or greater than about 95% sequence identity, to the GmCPD1 amino acid sequence (SEQ ID NO:8) or to the GmCPD2 amino acid sequence (SEQ ID NO:7). A polypeptide can have about 95% or greater sequence identity to each of domain A, domain B, and the heme-binding domain of GmCPD1. A polypeptide can have about 98% or about 99% or greater sequence identity to domain A of GmCPD1. A polypeptide can have about 95% or greater sequence identity to domain B of GmCPD1. A polypeptide can have about 95% or greater sequence identity to the heme-binding domain of GmCPD1. A polypeptide can include the amino acid sequence of GmCPD1 as set forth in SEQ ID NO:8. A polypeptide can include the amino acid sequence of GmCPD2 as set forth in SEQ ID NO:7. In certain cases, the polypeptide has the GmCPD1 sequence set forth in SEQ ID NO:8, or the GmCPD2 sequence set forth in SEQ ID NO:7.

An isolated polynucleotide can include a control element operably linked to a nucleic acid encoding a polypeptide described herein. A control element can be, without limitation, a tissue-specific promoter, an inducible promoter, a constitutive promoter, or a broadly expressing promoter. The control element can regulate, for example, expression of a polypeptide in the leaf, stem, and roots of an Arabidopsis plant. An Arabidopsis plant, when expressing a polypeptide described herein, can exhibit a height at least about 7% greater than an Arabidopsis plant not expressing the polypeptide.

Also provided are recombinant vectors, which can include any of the polynucleotides described herein, and (ii) a control element operably linked to the polynucleotide wherein a polypeptide coding sequence in the polynucleotide can be transcribed and translated in a host cell. Host cells comprising such recombinant vectors are also provided.

In another aspect, transgenic plants are provided. For example, a transgenic plant can include at least one exogenous polynucleotide comprising a nucleic acid encoding a polypeptide having (a) about 80% or greater sequence identity to the GmCPD1 amino acid sequence set forth in SEQ ID NO:8
(b) about 90% or greater sequence identity to each of domain A, domain B, and the heme-binding domain of GmCPD1; and
(c) about 80% or greater sequence identity to domain C of GmCPD1.

A plant can be a monocot, a dicot, or a gymnosperm. The polypeptide can be effective for catalyzing the hydroxylation of 6-deoxocathasterone at C-23 to produce 6-deoxoteasterone.

In another aspect, a method for producing a transgenic plant is provided that comprises:
(a) introducing a polynucleotide described herein into a plant cell to produce a transformed plant cell; and
(b) producing a transgenic plant from the transformed plant cell. A transgenic plant can have an altered phenotype relative to a wild-type plant. An altered phenotype can be increased plant height. An altered phenotype can be an increased amount of 6-deoxoteasterone.

In another embodiment, a method of modulating a BL biosynthetic pathway in a plant is provided that includes:
(a) producing a transgenic plant containing an exogenous polynucleotide as described herein; and
(b) culturing the transgenic plant under conditions wherein a polynucleotide is expressed. A modulation can be an increased amount of 6-deoxoteasterone.

Isolated polypeptides are also provided. An isolated polypeptide can have:
(a) about 80% or greater sequence identity to the GmCPD1 amino acid sequence set forth in SEQ ID NO:8;
(b) about 90% or greater sequence identity to each of domain A, domain B, and the heme-binding domain of GmCPD1; and
(c) about 80% or greater sequence identity to domain C of GmCPD1.

An isolated polypeptide can be effective for catalyzing the hydroxylation of 6-deoxocathasterone at C-23 to produce 6-deoxoteasterone. An isolated polypeptide can include, for example, the GmCPD1 amino acid sequence as set forth in SEQ ID NO:8; the GmCPD2 amino acid sequence as set forth in SEQ ID NO:7; the Corn CPD amino acid sequence (SEQ ID NO:5) as set forth in the Alignment Table, or the Rice CPD amino acid sequence (SEQ ID NO:6) as set forth in the Alignment Table.

In another aspect, an isolated polynucleotide provided herein can include a nucleic acid encoding a polypeptide having about 85% or greater (e.g., about 90% or greater or about 95% or greater) sequence identity to an amino acid sequence set forth in the Alignment Table, e.g., SEQ ID NOS:9, 17, 5, 6, 15, 14, 2, 7, 8, or 18. An isolated polynucleotide can include a nucleic acid encoding a polypeptide having about 85% or greater (e.g., about 90% or greater or about 95% or greater) sequence identity to an amino acid sequence set forth in the Alignment Table, wherein the amino acid sequence is selected from the Corn CPD (SEQ ID NO:5), Rice CPD (SEQ ID NO:6), Soy1 CPD (SEQ ID NO:8), and Soy2 CPD (SEQ ID NO:7) amino acid sequences. A recombinant vector can include a described polynucleotide and a control element operably linked to the polynucleotide. A host cell can include such a recombinant vector. A control element can be a promoter. A promoter can be, without limitation, a tissue-specific promoter, an inducible promoter, a constitutive promoter, or a broadly-expressing promoter.

In another aspect, a transgenic plant that includes at least one exogenous polynucleotide is provided, where the at least one exogenous polynucleotide includes a nucleic acid encoding a polypeptide:
(a) having about 85% or greater sequence identity to an amino acid sequence set forth in the Alignment Table; or
(b) corresponding to the Consensus Sequence set forth in the Alignment Table. The exogenous polynucleotide can further comprise a control element operably linked to the nucleic acid encoding the polypeptide. A control element can be a promoter. A promoter can be, without limitation, a tissue-specific promoter, an inducible promoter, a constitutive promoter, or a broadly-expressing promoter. A transgenic plant can exhibit an altered phenotype relative to a control plant, such as an increased height. A plant can be a monocot, or a dicot, or a gymnosperm. A polypeptide can be effective for catalyzing the hydroxylation of 6-deoxocathasterone at C-23 to produce 6-deoxoteasterone. Seed of any of the transgenic plants described herein are also contemplated.

In a further aspect, a method of modulating the height of a plant is provided which includes a) introducing into a plant cell an exogenous nucleic acid comprising a polynucleotide sequence encoding a polypeptide having 80% or greater sequence (e.g., 85% or greater, identity to an amino acid sequence set forth in the Alignment Table, where a plant produced from said plant cell has a different height as compared to a corresponding control plant that does not comprise said exogenous nucleic acid, and where the exogenous nucleic acid further comprises a broadly expressing promoter operably linked to the polynucleotide.

In another embodiment, a method of modulating the height of a plant includes:

a) introducing into a plant cell an exogenous nucleic acid comprising a polynucleotide sequence encoding a polypeptide having 80% or greater (e.g., 85% or greater, 90% or greater, 95% or greater) sequence identity to an amino acid sequence set forth in the Alignment Table, where a plant produced from the plant cell has different height as compared to a corresponding control plant that does not comprise said exogenous nucleic acid, and where the amino acid sequence is an amino acid sequence set forth in the Alignment Table other than the Arabidopsis amino acid sequence. The plant can be a monocot, dicot, or gymnosperm. A modulation can be an increase in height.

In another aspect, an isolated polypeptide having about 85% or greater sequence identity to an amino acid sequence set forth in the Alignment Table, where said amino acid sequence is selected from the Corn CPD, Rice CPD, Soy1 CPD, and Soy2 CPD amino acid sequences, is provided.

A transgenic plant comprising at least one exogenous polynucleotide is also provided, where the at least one exogenous polynucleotide comprises a nucleic acid encoding a polypeptide having about 85% or greater (e.g., about 90% or greater, about 95% or greater) sequence identity to an amino acid sequence set forth in the Alignment Table, and where the amino acid sequence is selected from the Corn CPD, Rice CPD, Soy1 CPD, and Soy2 CPD amino acid sequences.

In another embodiment, a method of modulating the height of a plant is provided that includes:

a) introducing into a plant cell an exogenous nucleic acid comprising a polynucleotide sequence encoding a polypeptide having 80% or greater (e.g., 85% or greater, 90% or greater, 95% or greater) sequence identity to an amino acid sequence set forth in the Alignment Table, wherein a plant produced from the plant cell has a different height as compared to a corresponding control plant that does not comprise the exogenous nucleic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an Alignment Table showing an amino acid sequence alignment of Arabidopsis CPD with orthologous CPD amino acid sequences; FIG. 1 also sets forth a Consensus Sequence, as described herein.

FIG. 2 demonstrates RT-PCR analysis of T3 GmCPD2 Plants. The plants are transgenic and wild-type segregants from transformation event ME0874 using primers that amplify actin (lanes 1-4) or GmCPD2 transcripts (5-8). Samples 1 and 5 are from ME0874-1-5, samples 4 and 8 are from ME0874-5-11, and samples 2 and 3 are from the wild-type segregants ME0874-1-8; samples 6 and 7 are from the wild-type segregants ME0874-5-6. RNA from 14 DAG seedlings was used for the RT-PCR.

FIG. 3 shows the phenotype of p32449:CPD Arabidopsis plants. FIG. 4A: T3 plants from transformation events ME01137 (ME01137-1-21 and ME01130-3-24) show increased height when compared with wild-type segregants (ME01137-1-5 and ME01137-3-8, control). FIG. 4B: Measurements of T3 plant height at 60 DAG (n>10). The measurements indicate that T3 plants from each of the two ME01137 lines were about 20% taller than wild-type segregants. The error bars represent single standard deviations.

FIG. 4 demonstrates the phenotype of p32449:GmCPD1 Arabidopsis plants. FIG. 4A: T3 plants from transformation event ME0819 (ME0819-3-3 and ME0819-1-6) show increased height when compared with wild-type segregants (ME0819-1-11 and ME0819-3-10, control). FIG. 4B: Measurements of T3 plant height at 30 DAG (upper panel, n=10) and at 60 DAG (lower panel, n=10). The measurements indicate that T3 plants from each of the two ME01137 lines were about 10% taller than wild-type segregants. The error bars represent single standard deviations. These data suggest that GmCPD1 is a functional homolog (ortholog) of CPD.

FIG. 5 demonstrates the phenotype of p32449:GmCPD2 Arabidopsis plants. FIG. 5A: T3 plants from transformation event ME0874. One segregant (ME0874-5-11) showed evidence of increased height when compared with wild-type segregants ME0874-5-6 and ME0874-1-8 (control), but a second segregant (ME0874-1-5) did not. FIG. 5B: Measurements of T3 plant heights, at maturity (˜68 DAG) (n=10). The error bars represent single standard deviations.

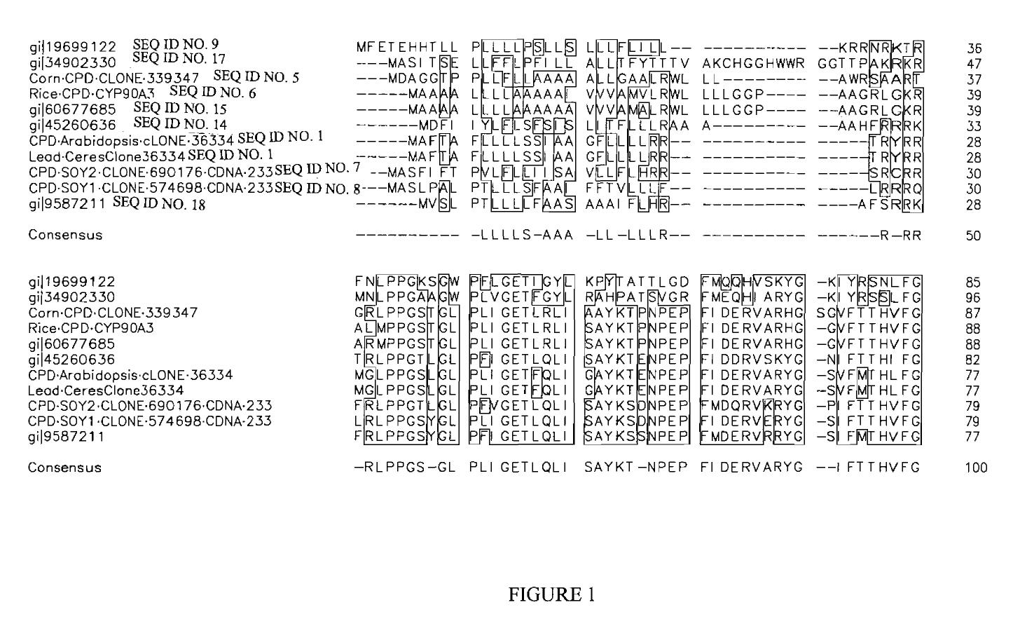

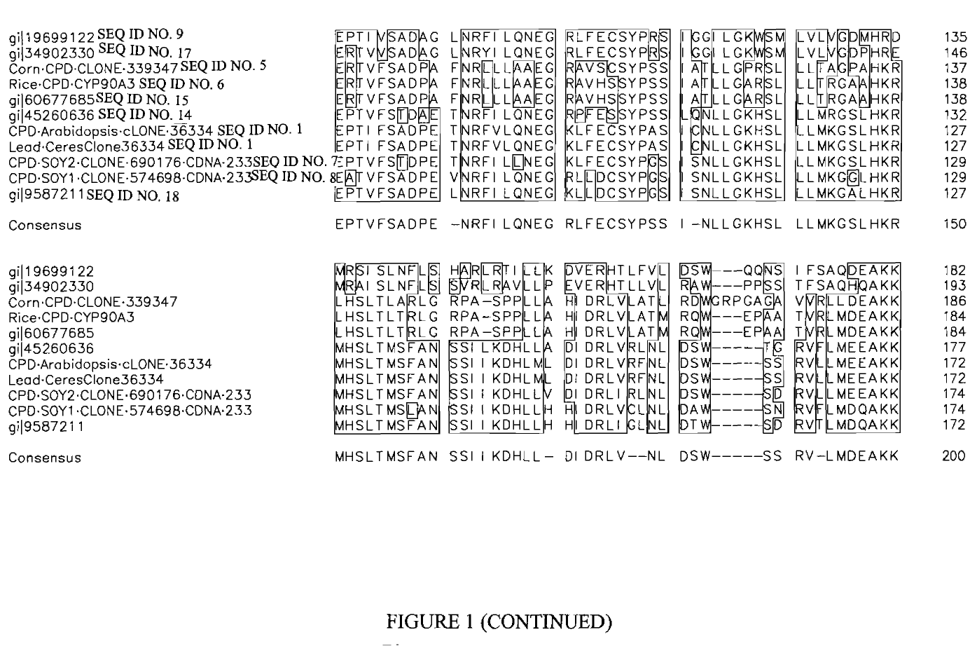

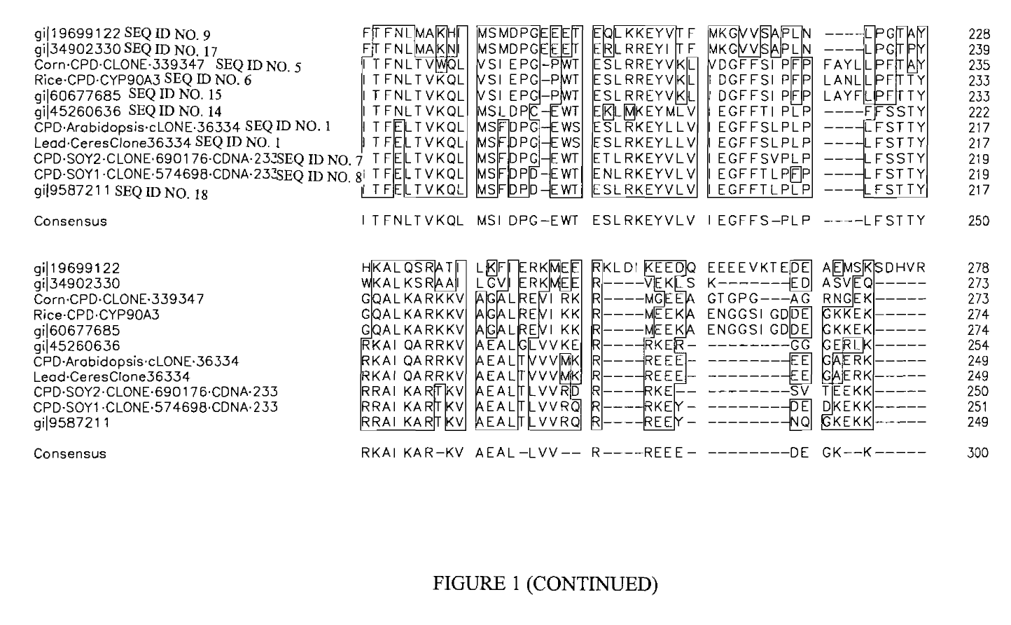

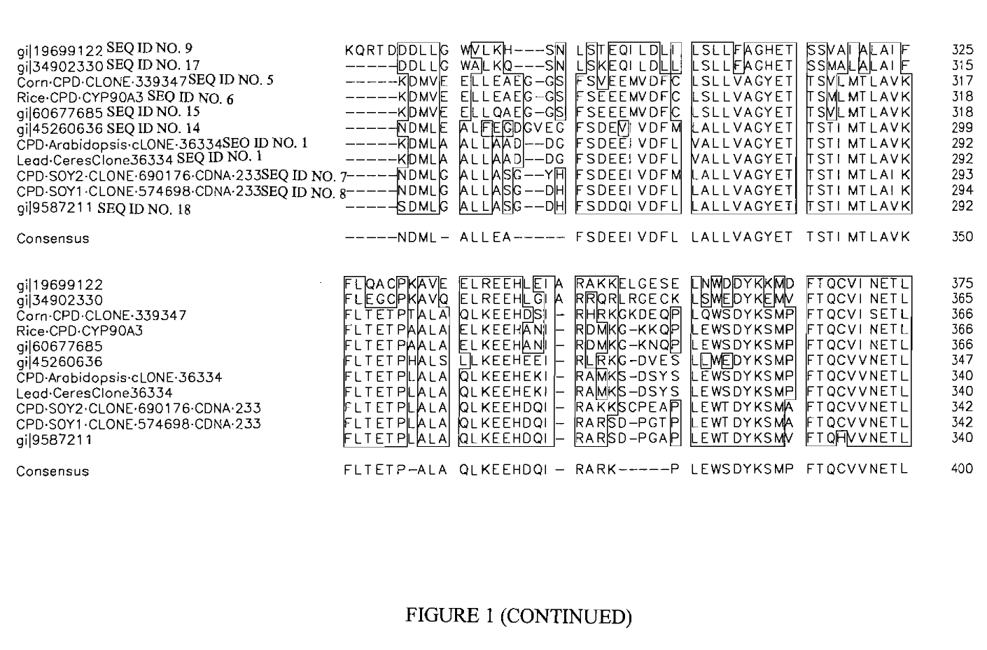

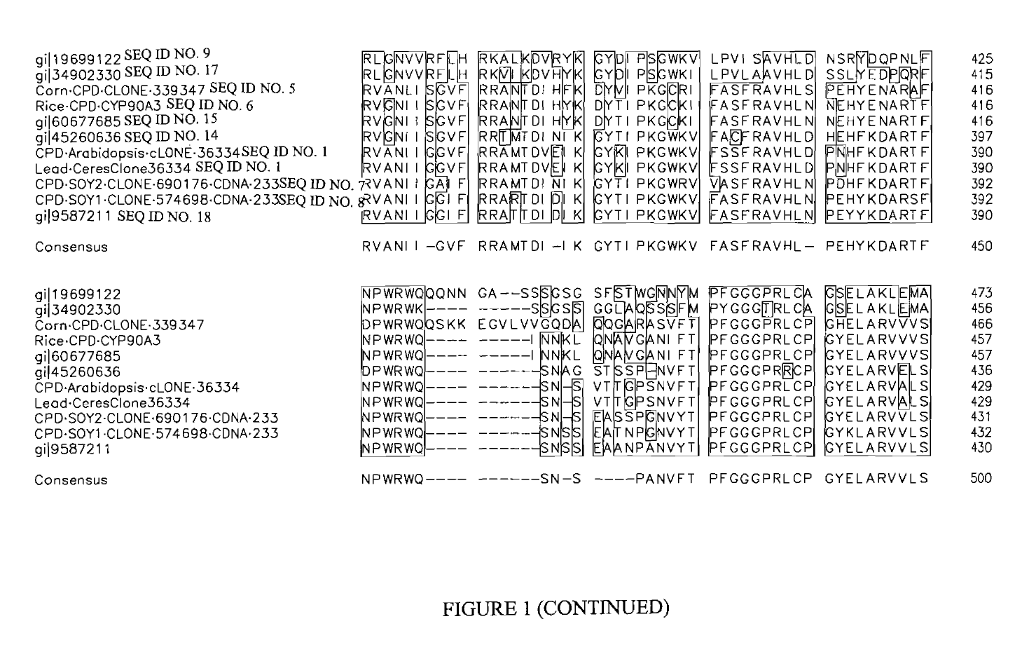

### DETAILED DESCRIPTION

Polynucleotides and Polypeptides

Polynucleotides and polypeptides described herein are of interest because when they are expressed non-naturally (e.g., with respect to: location in a plant, such as root vs. stem; environmental or developmental condition; plant species; time of development; and/or in an increased or decreased amount), they can produce plants with increased height and/or biomass. Thus, the polynucleotides and polypeptides are useful in the preparation of transgenic plants having particular application in the agricultural and forestry industries.

In particular, isolated P₄₅₀ polynucleotide and polypeptide sequences, including polynucleotide sequence variants, fusions, and fragments, are provided. An isolated P₄₅₀ polynucleotide or polypeptide can be an ortholog to a cpd polynucleotide or CPD polypeptide. Thus, isolated cpd polynucleotide and CPD polypeptide sequences, including orthologous CPD polypeptides to Arabidopsis CPD, are described herein.

CPD is a cytochrome P₄₅₀ polypeptide that, among other activities, catalyzes the hydroxylation of 6-deoxocathasterone at C-23 to produce 6-deoxoteasterone, an enzymatic step immediately downstream from the oxidation at C-22 by DWF4, another cytochrome P₄₅₀ protein. Thus, a polypeptide sequence can exhibit a biochemical activity or affect a plant phenotype in a manner similar to a CPD polypeptide and represents an orthologous polypeptide to the Arabidopsis CPD protein.

The terms “nucleic acid” or “polynucleotide” are used interchangeably herein, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus, or the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. Examples of means by which this can be accomplished in plants are well known in the art, such as Agrobacterium-mediated transformation (for dicots, see Salomon et al. EMBO J. 3:141 (1984); Herrera-Estrella et al. EMBO J. 2:987 (1983); for monocots, see Escudero et al., Plant J. 10:355 (1996), Ishida et al., Nature Biotechnology 14:745 (1996), May et al., Bio/Technology 13:486 (1995)); biolistic methods (Armaleo et al., Current Genetics 17:97 1990)); electroporation; in planta techniques, and the like. Such a plant containing an exogenous nucleic acid is referred to here as a T₁ plant for the primary transgenic plant, a T₂ plant for the first generation, and T₃, T₄, etc. for second and subsequent generation plants. T₂ progeny are the result of self-fertilization of a T₁ plant. T₃ progeny are the result of self-fertilization of a T₂ plant.

An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell (or plant) under consideration. For example, a transgenic plant containing an exogenous nucleic acid can be the progeny of a cross between a stably transformed plant and a non-transgenic plant. Such progeny are considered to contain the exogenous nucleic acid.

The term “polypeptide” as used herein refers to a compound of two or more subunit amino acids, amino acid analogs, or other peptidomimetics, regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers. Full-length proteins, analogs, mutants, and fragments thereof are encompassed by this definition.

By “isolated” or “purified” with respect to a polypeptide it is meant that the polypeptide is separated to some extent from the cellular components with which it is normally found in nature (e.g., other polypeptides, lipids, carbohydrates, and nucleic acids). An purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

Isolated polynucleotides can include nucleic acids that encode cytochrome P₄₅₀ polypeptides. An encoded polypeptide can be a member of the CPD P₄₅₀ subfamily. A polypeptide encoded by a polynucleotide and/or nucleic acid described herein can exhibit greater than 55% (e.g., greater than 57, 60, 65, 70, 71, 72, 73, 74, 75, 76, 77, 78, 79, 80, 81, 82, 84, 85, 86, 87, 88, 90, 92, 94, 95, 97, 98, or 99%) sequence identity to the Arabidopsis CPD amino acid sequence (SEQ ID NO:2) (also identified as Ceres Clone 36334 herein). In some cases, a polypeptide encoded by a polynucleotide described herein can exhibit up to 76% sequence identity to the Arabidopsis CPD amino acid sequence, e.g., about 40%, 50%, 55%, 59%, 60%, 61%, 63%, 65%, 68%, 70%, 72%, or 75% sequence identity. In certain cases, a polypeptide encoded by a polynucleotide described herein can exhibit 80% or more sequence identity to the Arabidopsis CPD amino acid sequence, e.g., 81%, 82%, 83%, 84%, 85%, 86%, 87%, 88%, 89%, 90%, 91%, 92%, 93%, 94%, 95%, 96%, 97%, 98%, 99% or 100% sequence identity.

The Alignment Table sets forth amino acid sequences of CPD orthologs and a Consensus Sequence. For example, the Alignment Tables provides the amino acid sequences, respectively, of two CPD homologs from soybean, GmCPD1 and GmCPD2 (SEQ ID NOs:8 and 7 respectively) (also identified in the Alignment Table as CPD SOY1 and CPD SOY2, respectively). The two soybean polypeptides were identified as CPD homologs as described below. GmCPD1 exhibits 77% sequence identity to Arabidopsis CPD at the amino acid level, while GmCPD2 exhibits 78% sequence identity to Arabidopsis CPD. Other orthologs are also set forth in the Alignment Table, including those from corn and rice.

In certain cases, therefore, an isolated polynucleotide can include a nucleic acid encoding a polypeptide having about 80% or greater sequence identity to an amino acid sequence set forth in the Alignment Table other than the Arabidopsis amino acid sequence, e.g., about 82, 85, 87, 90, 92, 95, 96, 97, 98, 99, or 100% sequence identity to such a sequence. For example, an isolated polynucleotide can include a nucleic acid encoding a polypeptide having about 80% or greater sequence identity to the SOY1 amino acid sequence, or the SOY2 amino acid sequence, or the Corn amino acid sequence, or the Rice amino acid sequence. As used herein, the term “percent sequence identity” refers to the degree of identity between any given query sequence and a subject sequence. A percent identity for any query nucleic acid or amino acid sequence, e.g., a CPD ortholog polypeptide, relative to another subject nucleic acid or amino acid sequence can be determined as follows. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw). To determine a “percent identity” between a query sequence and a subject sequence, the number of matching bases or amino acids in the alignment is divided by the total number of matched and mismatched bases or amino acids, followed by multiplying the result by 100.

It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

A consensus amino acid sequence for a CPD ortholog polypeptide can be determined by aligning amino acid sequences (e.g., amino acid sequences set forth in the Alignment Table) from a variety of plant species and determining the most common amino acid or type of amino acid at each position. For example, a consensus sequence can be determined by aligning the Arabidopsis CPD amino acid sequence with orthologous amino acid sequences, as shown in the Alignment Table.

Other means by which CPD ortholog polypeptides can be identified include functional complementation of CPD polypeptide mutants. Suitable CPD ortholog polypeptides also can be identified by analysis of nucleotide and polypeptide sequence alignments. For example, performing a query on a database of nucleotide or polypeptide sequences can identify orthologs of the Arabidopsis CPD polypeptide. Sequence analysis can involve BLAST or PSI-BLAST analysis of nonredundant databases using amino acid sequences of known methylation status polypeptides. Those proteins in the database that have greater than 40% sequence identity can be candidates for further evaluation for suitability as CPD orthologous polypeptides. If desired, manual inspection of such candidates can be carried out in order to narrow the number of candidates to be further evaluated. Manual inspection can be performed by selecting those candidates that appear to have domains suspected of being present in CPD orthologous polypeptides.

Typically, conserved regions of CPD orthologous polypeptides exhibit at least 40% amino acid sequence identity (e.g., at least 45%, at least 50%, at least 60%, at least 70%, at least 80%, or at least 90% amino acid sequence identity). Conserved regions of target and template polypeptides can exhibit at least 92%, 94%, 96%, 98%, or 99% amino acid sequence identity. Amino acid sequence identity can be deduced from amino acid or nucleotide sequences. In certain cases, highly conserved domains can be identified within CPD orthologous polypeptides. These conserved regions can be useful in identifying other orthologous polypeptides.

Domains are groups of contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed.

The identification of conserved regions in a template, or subject, polypeptide can facilitate production of variants of CPD or CPD orthologous polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Pfam/ and online at genome.wustl.edu/Pfam/. Descriptions of the information included at the Pfam database are included in Sonnhammer et al., 1998, Nucl. Acids Res. 26: 320-322; Sonnhammer et al., 1997, Proteins 28:405-420; and Bateman et al., 1999, Nucl. Acids Res. 27:260-262. From the Pfam database, consensus sequences of protein motifs and domains can be aligned with the template polypeptide sequence to determine conserved region(s).

By taking advantage of the relationship between sequence, structure, and function that is characteristic of cytochrome P₄₅₀ proteins in general and C-23 hydroxylases in particular, orthologous functionally comparable polypeptides to CPD are provided. Cytochrome P₄₅₀ proteins include a number of domains characterized by functional and/or structural characteristics. (See U.S. Ser. No. 09/502,426, filed Feb. 11, 2000, entitled “Dwf4 Polynucleotides, Polypeptides, and Uses Thereof,” incorporated by reference herein; Nelson et al., Pharmacogenetics, Vol. 6(1):1-42, Feb. 1996; and Paquette et al., DNA and Cell Biology, Vol. 19(5):307-317 (2000)). Domains A, B, C, and the heme-binding domain play important roles in P₄₅₀ enzymatic function. Domain A is known as the substrate and oxygen (O₂) binding domain, while Domain B is known as the steroid-binding domain. The function of Domain C has not yet been fully characterized.

As cytochrome P₄₅₀ and C-23 hydroxylase proteins include these separate functional and/or structural domains, a polypeptide of the invention can demonstrate various percentage amounts of sequence identity over a defined length of the molecule, e.g., over one or more domains relative to GmCPD1 or GmCPD2, or the corn CPD, or the rice CPD. Variations in the amount of sequence identity of a polypeptide in one or more domains can yield other orthologous CPD polypeptides. For example, certain polypeptides can have a high degree of sequence identity in one or more domains of interest. Accordingly, in certain cases, a polypeptide can include any combination of domains having particular values of sequence identity to one or more of the corresponding domains in a reference polypeptide (e.g., CPD, GmCPD1, GmCPD2, corn CPD, rice CPD), provided that the polypeptide exhibits at least about 80% sequence identity (e.g., at least about 85, 90, 92, 95, 96, 97, 98, 99 or 100% sequence identity) to GmCPD1 or GmCPD2. Thus, a polypeptide having at least 80% sequence identity to GmCPD1 can exhibit, for example, 95% sequence identity to domain A of GmCPD1, 90% sequence identity to domain B of GmCPD2, 95% sequence identity to domain C of CPD, and 99% sequence identity to the heme-binding domain of GmCPD1.

In certain cases, a polypeptide of the invention can exhibit about 90% or greater (e.g., about 91, 92, 93, 94, 95, 96, 97, 98, 99, or 100%) sequence identity, independently, to one or more of domains A, B, and the heme-binding domain of GmCPD1. Alternatively, a polypeptide can exhibit about 90% or greater (e.g., about 91, 92, 93, 94, 95, 96, 97, 98, 99, or 100%) sequence identity, independently, to one or more of domains A, B, and the heme-binding domain of GmCPD2. In yet other cases, a polypeptide can exhibit about 80% or greater (e.g., about 85, 90, 92, 95, 96, 97, 98, 99 or 100%) sequence identity to domain C of GmCPD1, or about 80% or greater (e.g., about 85, 90, 92, 95, 96, 97, 98, 99 or 100%) sequence identity to domain C of GmCPD2.

In certain cases, a polypeptide described herein can be orthologous to CPD as determined by it performing at least one of the biochemical activities of CPD or affecting a plant phenotype in a similar manner to CPD. Thus, a polypeptide can catalyze a similar reaction as CPD or affect a plant phenotype in a manner similar to CPD. For example, CPD is known to catalyze the hydroxylation of 6-deoxocathasterone at C-23 to produce 6-deoxoteasterone. A polypeptide of the invention may also perform the same enzymatic step. In certain cases, an orthologous CPD polypeptide exhibits at least 60% of the biochemical activity of the native protein, e.g., at least 70%, 80%, 90%, 95%, or even more than 100% of the biochemical activity. Methods for evaluating biochemical activities are known to those having ordinary skill in the art, and include enzymatic assays, radiotracer assays, etc.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

### Recombinant Constructs, Vectors and Host Cells

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The terms “regulatory sequence,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory sequences that can reside within coding sequences, such as secretory signals and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into mRNA, which then can be translated into the protein encoded by the coding sequence. Thus, a regulatory region can modulate, e.g., regulate, facilitate or drive, transcription in the plant cell, plant, or plant tissue in which it is desired to express a nucleic acid encoding a tocopherol-modulating polypeptide.

A promoter is an expression control sequence composed of a region of a DNA molecule, typically within 100 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoter regions to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. Alternatively, constitutive promoters can promote transcription of an operably linked nucleic acid in most or all tissues of a plant, throughout plant development. Other classes of promoters include, but are not limited to, inducible promoters, such as promoters that confer transcription in response to an external stimuli such as chemical agents, developmental stimuli, or environmental stimuli.

In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are essentially specific to seeds (“seed-preferential promoters”) can be useful. Seed-specific promoters can promote transcription of an operably linked nucleic acid in endosperm and cotyledon tissue during seed development.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

An “inducible promoter” refers to a promoter that is regulated by particular conditions, such as light, anaerobic conditions, temperature, chemical concentration, protein concentration, conditions in an organism, cell, or organelle. A cell type or tissue-specific promoter can drive expression of operably linked sequences in tissues other than the target tissue. Thus, as used herein a cell-type or tissue-specific promoter is one that drives expression preferentially in the target tissue, but can also lead to some expression in other cell types or tissues as well. Methods for identifying and characterizing promoter regions in plant genomic DNA are known.

In certain cases, a broadly expressing promoter can be included. For example, broadly expressing promoters such as p326, p32449, p13879, YP0050, YP0144, and YP0190 can be used. A promoter can be said to be “broadly expressing” as used herein when it promotes transcription in many, but not all, plant tissues. For example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. In certain cases, a broadly expressing promoter operably linked to a sequence can promote transcription of the linked sequence in a plant shoot at a level that is at least two times (e.g., at least 3, 5, 10, or 20 times) greater than the level of transcription in root tissue or a developing seed. In other cases, a broadly expressing promoter can promote transcription in a plant shoot at a level that is at least two times (e.g., at least 3, 5, 10, or 20 times) greater than the level of transcription in a reproductive tissue of a flower.

In such cases, a polynucleotide operably linked to a broadly expressing promoter can be any of the polynucleotides described above, e.g., encoding an amino acid sequence as set forth in the Alignment Table, or a polynucleotide including a nucleic acid sequence encoding a polypeptide exhibiting at least about 80% (e.g., at least about 82%, 85%, 86%, 87%, 90%, 92%, 95%, 96%, 97%, 98%, 99% or 100%) sequence identity to one or more of such amino acid sequences. In cases where a constitutive promoter such as 35S is employed, a polynucleotide can include a nucleic acid encoding a polypeptide having 85% or greater sequence identity to an amino acid sequence set forth in an Alignment Table other than the Arabidopsis CPD amino acid sequence (e.g., about 86, 87, 90, 92, 95, 96, 97, 98, 99, or 100% sequence identity), or can include a nucleic acid encoding a polypeptide corresponding to the consensus sequence for a CPD polypeptide set forth in the Alignment Table.

Non-limiting examples of promoters that can be included in the nucleic acid constructs provided herein include the cauliflower mosaic virus (CaMV) 35S transcription initiation region, the 1′ or 2′ promoters derived from T-DNA of Agrobacterium tumefaciens, promoters from a maize leaf-specific gene described by Busk [(1997) Plant J., 11:1285-1295], kn1-related genes from maize and other species, transcription initiation regions from various plant genes such as the maize ubiquitin-1 promoter, and promoters set forth in U.S. Patent Application Ser. Nos. 60/505,689; 60/518,075; 60/544,771; 60/558,869; 60/583,691; 60/619,181; 60/637,140; 10/957,569; 11/058,689; 11/172,703 and PCT/US05/23639, e.g., promoters designated YP0086 (gDNA ID 7418340), YP0188 (gDNA ID 7418570), YP0263 (gDNA ID 7418658), p13879, p326, p32449 (SEQ ID NO:19), YP0050, YP0144, YP0190, PT0758; PT0743; PT0829; YP0096 and YP0119.

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences.

A polyadenylation region at the 3′-end of a coding region can also be operably linked to a coding sequence. The polyadenylation region can be derived from the natural gene, from various other plant genes, or from an Agrobacterium T-DNA gene.

The vectors provided herein also can include, for example, origins of replication, scaffold attachment regions (SARs), and/or markers. A marker gene can confer a selectable phenotype on a plant cell. For example, a marker can confer, biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The recombinant DNA constructs provided herein typically include a polynucleotide sequence (e.g., a sequence encoding a CPD or CPD orthologous polypeptide) inserted into a vector suitable for transformation of plant cells. Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

### Transgenic Plants and Cells

The vectors provided herein can be used to transform plant cells and, if desired, generate transgenic plants. Thus, transgenic plants and plant cells containing the nucleic acids described herein also are provided, as are methods for making such transgenic plants and plant cells. A plant or plant cells can be transformed by having the construct integrated into its genome, i.e., can be stably transformed. Stably transformed cells typically retain the introduced nucleic acid sequence with each cell division. Alternatively, the plant or plant cells also can be transiently transformed such that the construct is not integrated into its genome. Transiently transformed cells typically lose some or all of the introduced nucleic acid construct with each cell division, such that the introduced nucleic acid cannot be detected in daughter cells after sufficient number of cell divisions. Both transiently transformed and stably transformed transgenic plants and plant cells can be useful in the methods described herein.

Typically, transgenic plant cells used in the methods described herein constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. Seeds produced by a transgenic plant can be grown and then selfed (or outcrossed and selfed) to obtain seeds homozygous for the nucleic acid construct.

Alternatively, transgenic plant cells can be grown in suspension culture, or tissue or organ culture, for production of secondary metabolites. For the purposes of the methods provided herein, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

Techniques for transforming a wide variety of higher plant species are known in the art. The polynucleotides and/or recombinant vectors described herein can be introduced into the genome of a plant host using any of a number of known methods, including electroporation, microinjection, and biolistic methods. Alternatively, polynucleotides or vectors can be combined with suitable T-DNA flanking regions and introduced into a conventional Agrobacterium tumefaciens host vector. Such Agrobacterium tumefaciens-mediated transformation techniques, including disarming and use of binary vectors, are well known in the art. Other gene transfer and transformation techniques include protoplast transformation through calcium or PEG, electroporation-mediated uptake of naked DNA, electroporation of plant tissues, viral vector-mediated transformation, and microprojectile bombardment (see, e.g., U.S. Pat. Nos. 5,538,880, 5,204,253, 5,591,616, and 6,329,571). If a cell or tissue culture is used as the recipient tissue for transformation, plants can be regenerated from transformed cultures using techniques known to those skilled in the art.

The polynucleotides and vectors described herein can be used to transform a number of monocotyledonous and dicotyledonous plants and plant cell systems, including dicots such as safflower, alfalfa, clover, soybean, coffee, lettuce, carrot, grape, strawberry, amaranth, rapeseed (high erucic acid and canola), broccoli, peas, peanut, tomato, potato, beans (including kidney beans, lima beans, dry beans, green beans), melon (e.g., watermelon, cantaloupe), peach, pear, apple, cherry, orange, lemon, grapefruit, plum, mango or sunflower, as well as monocots such as oil palm, date palm, sugarcane, banana, sweet corn, popcorn, field corn, wheat, rye, barley, oat, onion, pineapple, rice, millet, sudangrass, switchgrass or sorghum. Gymnosperms such as fir, spruce and pine can also be suitable.

Thus, the methods and compositions described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods and compositions described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales, or with plants belonging to Gymnospermae, e.g., Pinales, Ginkgoales, Cycadales and Gnetales.

The methods and compositions can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vitis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant can be identified and isolated by selecting or screening the engineered plant material for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, SI RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. After a polynucleotide is stably incorporated into a transgenic plant, it can be introduced into other plants using, for example, standard breeding techniques.

Transgenic plants (or plant cells) can have an altered phenotype as compared to a corresponding control plant (or plant cell) that either lacks the transgene or does not express the transgene. A polypeptide can affect the phenotype of a plant (e.g., a transgenic plant) when expressed in the plant, e.g., at the appropriate time(s), in the appropriate tissue(s), or at the appropriate expression levels. Phenotypic effects can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10% (e.g., less than 9%, 8%, 7%, 6%, 5%, 4%, 3%, 2%, 1%, 0.5%, 0.1%, 0.01%, or 0.001%) of the amount of polypeptide or mRNA encoding the polypeptide exhibited by the plant of interest. Expression can be evaluated using methods including, for example, RT-PCR, Northern blots, SI RNAse protection, primer extensions, Western blots, protein gel electrophoresis, immunoprecipitation, enzyme-linked immunoassays, chip assays, and mass spectrometry. It should be noted that if a polypeptide is expressed under the control of a tissue-specific or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

A phenotypic effect can be increased plant height, biomass, and cell length. For example, when a polypeptide described herein is expressed in a transgenic plant, the transgenic plant can exhibit a height at least about 7% greater (e.g., at least about 10%, 15%, 20%, 25%, 30%, 35%, 50%, 75%, 90%, 95% or more) than a plant not expressing the polypeptide. It should be noted that phenotypic effects are typically evaluated for statistical significance by analysis of multiple experiments, e.g., analysis of a population of plants or plant cells, etc. It is understood that when comparing phenotypes to assess the effects of a polypeptide, a statistically significant difference indicates that that particular polypeptide warrants further study. Typically, a difference in phenotypes is considered statistically significant at p≦0.05 with an appropriate parametric or non-parametric statistic, e.g., Chi-square test, Student's t-test, Mann-Whitney test, or F-test.

Other phenotypic effects can be evaluated by methods known to those of ordinary skill in the art, including cell length measurements at specific times in development; measurements of BL usage; sterol detection assays; detection of reaction products or by-products; and dose-response tests on putative enzymatic substrates. See, for example, U.S. Ser. No. 09/502,426.

Altering Expression Levels of P₄₅₀ Polypeptides

Overexpression

As described previously, the polynucleotides, recombinant vectors, host cells, and transgenic plants described herein can be engineered to yield overexpression of a polypeptide of interest. Overexpression of the polypeptides of the invention can be used to alter plant phenotypic characteristics relative to a control plant not expressing the polypeptides, such as to increase plant height. In addition, polypeptides can be overexpressed in combination with other polypeptides, e.g., other P₄₅₀ proteins or proteins involved in the BL biosynthetic pathway, such as DWF4. Such co-expression of polypeptides can result in additive or synergistic effects on a plant biochemical activity (e.g., enzymatic activity) or phenotype (e.g., height). Fusion polypeptides can also be employed and will typically include a polypeptide described herein fused in frame with another polypeptide, such as a polypeptide involved in BL biosynthesis (e.g., DWF4).

Inhibition of Expression

Alternatively, the polynucleotides and recombinant vectors described herein can be used to suppress or inhibit expression of an endogenous P₄₅₀ protein, such as CPD, in a plant species of interest. For example, inhibition or suppression of cpd transcription or translation may yield plants having increased shade tolerance.

A number of methods can be used to inhibit gene expression in plants. Antisense technology is one well-known method. In this method, a nucleic acid segment from the endogenous gene is cloned and operably linked to a promoter so that the antisense strand of RNA is transcribed. The recombinant vector is then transformed into plants, as described above, and the antisense strand of RNA is produced. The nucleic acid segment need not be the entire sequence of the endogenous gene to be repressed, but typically will be substantially identical to at least a portion of the endogenous gene to be repressed. Generally, higher homology can be used to compensate for the use of a shorter sequence. Typically, a sequence of at least 30 nucleotides is used (e.g., at least 40, 50, 80, 100, 200, 500 nucleotides or more). Thus, for example, an isolated nucleic acid provided herein can be an antisense nucleic acid to one of the aforementioned nucleic acids encoding a CPD polypeptide, e.g., the CPD orthologs set forth in the Alignment Table. Alternatively, the transcription product of an isolated nucleic acid can be similar or identical to the sense coding sequence of a CPD polypeptide, but is an RNA that is unpolyadenylated, lacks a 5′ cap structure, or contains an unsplicable intron.

Catalytic RNA molecules or ribozymes can also be used to inhibit expression. Ribozymes can be designed to specifically pair with virtually any target RNA and cleave the phosphodiester backbone at a specific location, thereby functionally inactivating the target RNA. The inclusion of ribozyme sequences within ribozymes confers RNA-cleaving activity upon them, thereby increasing their suppression activity. Methods for designing and using target RNA-specific ribozymes are known to those of skill in the art. See, generally, WO 02/46449 and references cited therein.

Methods based on RNA interference (RNAi) can also be used. RNA interference is a cellular mechanism to regulate the expression of genes and the replication of viruses. This mechanism is mediated by double-stranded small interfering RNA molecules (siRNA). A cell responds to a foreign double-stranded RNA (e.g., siRNA) introduced into the cell by destroying all internal mRNA containing the same sequence as the siRNA. Methods for designing and preparing siRNAs to target a target mRNA are known to those of skill in the art; see, e.g., WO 99/32619 and WO 01/75164. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of the CPD polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

Chemical synthesis, in vitro transcription, siRNA expression vectors, and PCR expression cassettes can then be used to prepare the designed siRNA.

Articles of Manufacture

The invention also provides articles of manufacture. Articles of manufacture can include one or more seeds from a transgenic plant described above. Typically, a substantially uniform mixture of seeds is conditioned and bagged in packaging material by means known in the art to form an article of manufacture. Such a bag of seed preferably has a package label accompanying the bag, e.g., a tag or label secured to the packaging material, a label printed on the packaging material, or a label inserted within the bag. The package label may indicate that plants grown from such seeds are suitable for making an indicated preselected polypeptide. The package label also may indicate that the seed contained therein incorporates transgenes that may provide desired phenotypic trains, such as increased height or shade tolerance to the plant.

### EXAMPLES

### Example 1

### Identification of CPD Orthologs

Two soybean polypeptides (and their corresponding cDNAs) were identified as CPD orthologs through polypeptide sequence comparisons (BLASTP analysis) of a library of soybean polypeptide sequences against a number of polypeptide databases, including a P₄₅₀, a plant, and a proprietary database. One clone (GmCPD1) is 77% identical to CPD and the other (GmCPD2) is 78% identical at the amino acid level, and both are greater than 80% identical to CPD within domains A—the O₂-binding domain, domain B—the steroid-binding domain, domain C, whose function is unknown, and the heme-binding domain [Kalb and Loper 1988]), as shown in Table 1. The numbers describe the homology (sequence identity) between CPD and soybean GmCPD1 and GmCPD2 at the amino acid level.

TABLE 1
Amino Acid Identities of Arabidopsis CPD and Two Soybean Proteins,
GmCPD1 and GmCPD2

| clone | Overall | A | B | C | Heme |
|---|---|---|---|---|---|
| GmCPD1 | 77% | 100.0% | 92.3% | 80.8% | 94.1% |
| GmCPD2 | 78% | 100.0% | 92.3% | 80.8% | 94.1% |

The two soybean clones are >80% identical and >85% similar to each other at the amino acid level. They are 100% identical to each other through domain A and 100.0% through domain B, as shown in FIG. 2 and Table 2. These domains represent the O₂-binding and steroid-binding domain of the CPD protein.

TABLE 2
Amino Acid Identity of Two Soybean CPD Homologs

| Overall | A | B | C | Heme |
|---|---|---|---|---|
| 81.1% | 100.0% | 100.0% | 84.6% | 95.5% |

### Example 2

### DNA Constructs, Transformation Experiments, and Transgenic Plant Lines

Promoter p32449 was operably linked to the following cDNA clones: CPD (clone 36334), GmCPD1 (clone 574698), and GmCPD2 (clone 690176). Promoter p32449 stimulates expression throughout epidermal and photosynthetic tissues in the shoot and in lateral and primary root tips. Ti plasmid vectors containing the P32449:DNA constructs were introduced into Arabidopsis plants using floral infiltration. The ecotype was WS. ME01137 lines contained p32449:CPD; ME0819 lines contained p32449:GmCPD1; and ME0874 lines contained p32449:GmCPD2. T2 segregants containing single T-DNA insertions were analyzed by PCR to test for the presence of p32449:CPD, p32449:GmCPD, and p32449:GmCPD2 in these lines.

Sequences of primers used to amplify the polynucleotides are as follows:

CPD (promoter to coding sequence):

| | |
|---|---|
| F CCTTATTCGTCTTCTTCGTTC | (SEQ ID NO:31) |
| R CAGACCCATCCGACGGTAAC | (SEQ ID NO:3) |

CPD (coding sequence to 3′ ocs transcription terminator):

| | |
|---|---|
| F CCCTTGGAGATGGCAGAGCA | (SEQ ID NO:4) |
| R TCATTAAAGCAGGACTCTAGC | (SEQ ID NO:32) |

GmCPD1 (promoter to coding sequence):

| | |
|---|---|
| F CCTTATTCGTCTTCTTCGTTC | (SEQ ID NO:31) |
| R CTACGTCAGAGAGTGCATTC | (SEQ ID NO:33) |

GmCPD1 (coding sequence to 3′ ocs transcription terminator):

| | |
|---|---|
| F GGGATCCAAAGTCTTTGCATC | (SEQ ID NO:34) |
| R TCATTAAAGCAGGACTCTAGC | (SEQ ID NO:32) |

GmCPD2 (promoter to coding sequence):

| | |
|---|---|
| F GGGATCCAAAGTCTTTGCATC | (SEQ ID NO:34) |
| R TTGTAAGCTGATATGAGCTG | (SEQ ID NO:35) |

T3 plants developed from the T2 lines that tested positive for the T-DNAs, and that were homozygous for them, were used for RT-PCR and phenotyping. CC2-4-4 lines contained p32449:DWF4. In these constructs, the DWF4 sequence was a gDNA sequence (Choe et al., 2001).

### Example 3

### Expression Detection (RT-PCR) and Phenotyping

Total RNA was isolated from seedlings 14 DAG, according to Qiagen™ protocols. RT-PCR was performed following the procedures recommended by Invitrogen Life Technologies. Reverse transcription was carried out using Superscript II RNase H reverse transcriptase. Primers in the coding sequence of GmCPD2 were used for amplifying GmCPD2 transcripts and had the following sequences:

| | |
|---|---|
| F1 ATGGCATCTTTCATCTTCAC | (SEQ ID NO:30) |
| R1 TTGTAAGCTGATATGAGCTG | (SEQ ID NO:35) |

Actin primers were used for the control, having the following sequences:

| | |
|---|---|
| ACT2-F: CGAGGGTTTCTCTCTTCCTC | (SEQ ID NO:28) |
| ACT2-R: TCTTACAATTTCCCGCTCTG | (SEQ ID NO:29) |

### Phenotyping

Putative phenotypes were noted at T1 and T2 generations. For lines showing putative T2 phenotypes, at least 10 T3 plants per T2 were scored for petiole length at 12 days after germination (DAG) and measured for rosette size at 30 DAG, for plant height at 60 DAG, and for shoot dry weight and seed weight at maturity (˜68 DAG). Wild-type T3 segregants were used as controls. For comparisons with T3 p32449:DWF4 plants, T3 CPD and GmCPD1 segregants and untransformed wild-types were used.

Plants were grown according to the following protocol in order to evaluate the phenotypic effects of polypeptides:

In a large container, mix 60% autoclaved SunshineMix #5 with 40% vermiculite. Add 2.5 tbsp of Osmocote, and 2.5 tbsp of 1% granular Marathon per 25 L of soil. Mix thoroughly with hands. Fill 1801 Deep 18 Pacs With Soil. Loosely fill 1801 Deep 18 pacs level to the rim with the prepared soil. Place filled pot into a utility flat with holes, within a no-hole utility flat. Repeat as necessary. One flat should contain 18 individual pots. Saturate soil and place flats on tables. Using a 400 ml water breaker, evenly water all pots in a “back and forth” motion until the soil is saturated and water is collecting in the bottom of the flats. If some pots are slightly dry, add about 1″ of water directly to the flat so that the soil will absorb the water from the bottom. After the soil is completely saturated, remove the excess water and plant the seed. Each flat will contain the progeny seed of one individual Ti plant. The progeny of 3 or more Ti events are usually planted (1 event=1 flat=18 pots). Place a single flat on the bench. Label the pots, e.g., break off barcoded ⅝″×5″ Styrene labeling tags and place one per pot. Choose the corresponding seed that matches the labeled flat/pots. Fold a single piece of 70 mm filter paper in half, and open it up so that there is a 90° angle. Pour ˜100 seeds onto the filter paper. Hold the filter paper with the thumb and middle finger. Sprinkle 3 or 4 seeds over each pot by gently tapping the filter paper with the index finger. It is important to place the seeds in the center of each pot because it will allow enough space for each plant to fully develop. Some practice may be required to skillfully accomplish this step. Repeat planting steps as necessary. Cover each flat with a propagation dome as it is finished. After sowing the seed for all the flats, place them into a dark 4° C. cooler. Keep the flats in the cooler for 2 nights for WS seed. Other ecotypes may require longer stratification. This cold treatment will help promote uniform germination of the seed. Remove flats from cooler. Place onto growth racks or benches. Cover the entire set of flats with 55% shade cloth. The cloth and domes should remain on the flats until the cotyledons have fully expanded. This usually takes about 4-5 days under standard greenhouse conditions. After the cotyledons have fully expanded, remove both the 55% shade cloth and propagation domes. Weed out excess seedlings. Segregating wild-type plants will be used as internal controls for quantitative and qualitative analysis. Using forceps, carefully weed out excess seedlings such that only one plant per pot exists throughout the flat. If no plants germinated for a particular pot, carefully transplant one of the excess seedlings as necessary to fill all 18 pots.

During the flowering stage of development, it is necessary to separate the individual plants so that they do not entwine themselves with other plants, causing cross-contamination and making seed collection very difficult. Place a Hyacinth stake in the soil next to the rosette, being careful not to damage the plant. Carefully wrap the primary and secondary bolts around the stake. Very loosely wrap a single plastic coated twist tie around the stake and the plant to hold it in place. Repeat staking process until all of the plants have been staked.

When senescence begins and flowers stop forming, stop watering. This will allow the plant to dry properly for seed collection. Before seed collection, pre-label 2.0 mL micro tubes with a barcode, common ID, box barcode, and location in box, and place into pre-labeled 100-place cryogenic storage boxes. Fold a clean piece of 8.5 inch×11 inch paper lengthwise and place on a table. Pull out and set aside the corresponding seed vial for the plant whose seed will be collected. Cut the base of the plant's bolts with scissors. Slowly remove the stake and the plant from the pot and place them over the paper. Carefully separate the stake from the plant, placing the stake in a container reserved for contaminated stakes. Run fingers along the bolts to shatter the siliques so that the seed falls onto the paper. Once all of the seed as been collected onto the paper, the plant can be disposed into a bio-waste container. Carefully fold the paper so that all of the seed collects in the crease of the paper. Use fingers to break open any intact siliques on the paper. Gently blow onto the seed in a sweeping manner in order to “clean” the seed of any excess plant material. Using the paper as a funnel, carefully pour the seed into the corresponding seed vial. Repeat seed collection steps as necessary until all seed has been collected.

The following measurements were taken:

Days to Bolt=number of days between sowing of seed and emergence of first inflorescence.

Number of Leaves=number of rosette leaves present at date of first bolt.

Rosette Area=Area of rosette at time of emergence of first inflorescence, using ((L×W)*3.14)/4.

Primary Inflorescence Thickness=diameter of primary inflorescence 2.5 cm up from base. This measurement was taken at the termination of flowering/onset of senescence.

Height=length of longest inflorescence from base to apex. This measurement was taken at the termination of flowering/onset of senescence.

### Results

### Expression of Transgenes

PCR was utilized to test for the presence of p32449:CPD, p32449:GmCPD, and p32449:GmCPD2 in T2 and T3 lines, and RT-PCR to demonstrate the expression of the transgenes in the T3 plants, as shown for ME0874-1-5, ME0874-5-11, and two wild-type segregants in FIG. 2. T3 plants that tested positive by RT-PCR were phenotyped.

### CPD Phenotypes

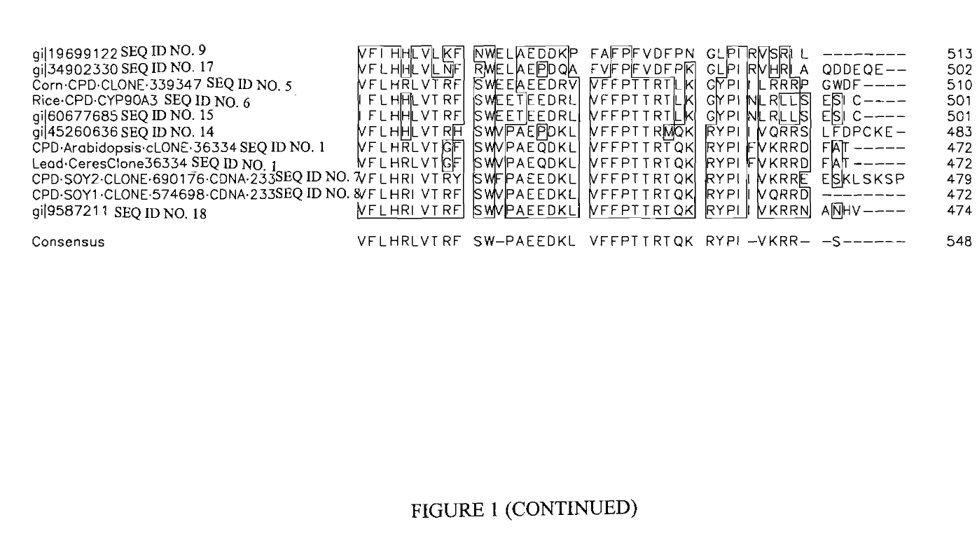

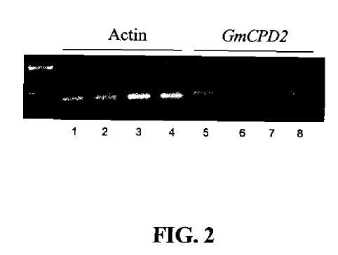

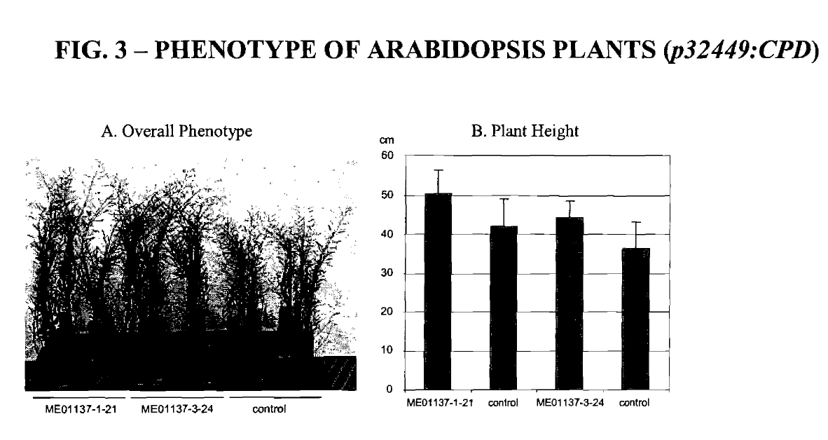

By studying T3 ME01137 plants that tested positive for expression of CPD by RT-PCR, and by comparing them with wild-type segregants (that tested negative), clear evidence of increased plant height was found, as shown in FIG. 3. Measurements indicated that T3 plants from each of ME01137-1-21 and 1130-3-24 were up to about 20% taller than the wild-type segregants ME01137-1-5 and ME01137-3-8. Standard t-test analysis showed that the variation in plant height was significant at the 0.05 level (P₁₁₃₀₋₁₋₂₁=0.038 and P₁₁₃₀₃₋₂₄=0.0018 for plants 60 DAG). Therefore, p32449-regulated expression of CPD can make Arabidopsis plants taller.

### GmCPD1 Phenotypes

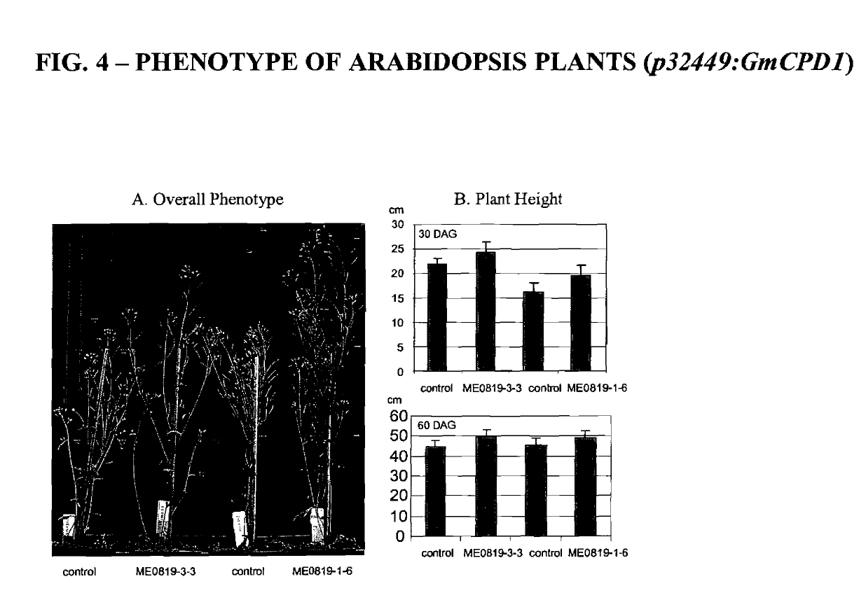

Phenotypes similar to those for CPD (ME01137) in T3 ME0819 lines containing p32449:GmCPD1 were observed. RT-PCR of ME0819-3-3 and ME0819-1-6 T3 plants showed that the transgenes were transcribed at a similar level in both lines (data not shown), and plants from both lines were taller than wild-type segregants, as shown in FIG. 4. Measurements indicated that T3 plants from each of two ME0819 lines (ME0819-1-6 and ME0819-3-3) were about 10% taller than the wild-type segregants ME0819-1-11 and ME0819-3-10, and t-test analysis showed that the variation was significant at the 0.05 level (P₀₈₁₉₋₁₋₆=0.0067, P₀₈₉₁₋₃₋₃=0.0019 for plants 30 DAG; P₈₁₉₋₁₋₆=0.0044, P₈₉₁₋₃₋₃=0.032 for 60 DAG plants.

### Expression of GmCPD2

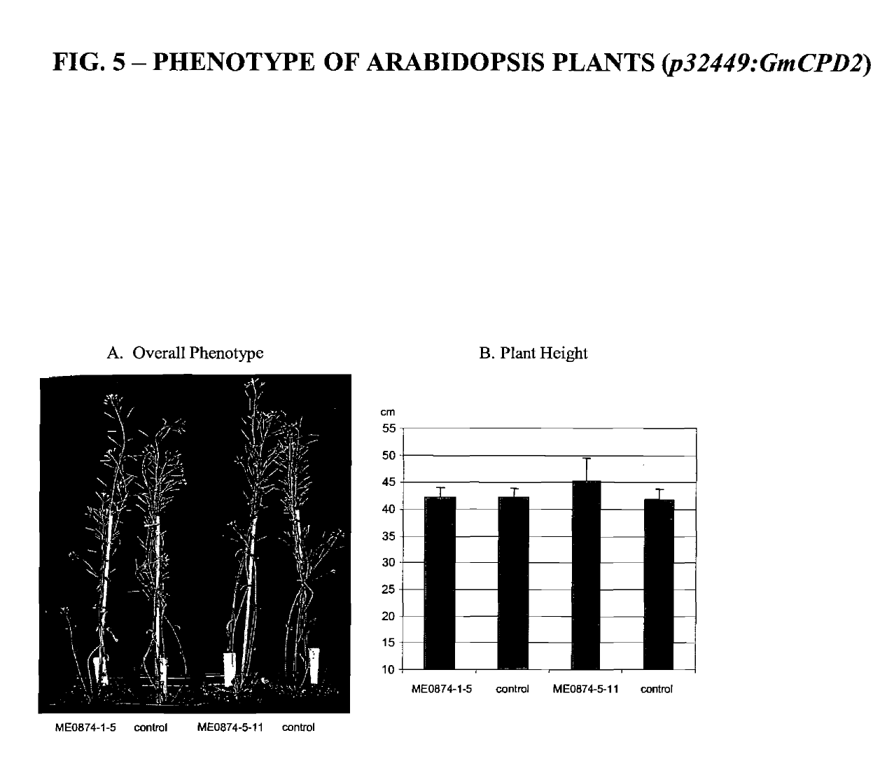

Phenotypes similar to those for CPD (ME01137) and p32449:GmCPD1 (ME0819) were observed in one T3 ME0874 line containing p32449:GmCPD2. Plants representing ME0874-5-11 were taller than wild-type segregants ME0874-5-6 and ME0874-1-8, as shown in FIG. 5. Measurement indicated that these T3 ME0874-5-11 plants were about 7% taller than wild-type segregants (FIG. 5), and t-test analysis showed that the variation was significant at the 0.05 level (P₈₇₄₋₅₋₁₁=0.041 for plants 30 DAG). However, whereas some ME0874-1-5 plants were also slightly taller than wild-type controls, such as the example in FIG. 5A, measurements of 10 such plants failed to reveal a consistent or significant increase in height (FIG. 5B). Since RT-PCR of ME0874-5-11 and ME0874-1-5 and plants showed that the transgenes were transcribed at a similar level in both lines (FIG. 2), it may be that larger sample sizes are needed to be certain of any growth and development differences between of ME0874-5-11 and ME0874-1-5.

### CPD and GmCPD1 Phenotypes Relative to DWF4 Phenotypes

Whereas CPD and GmCPD1 transgenes had clear effects on plant height, they did not result in seedling phenotypes. For example, whereas T3 p32449:DWF4 transgenes stimulated petiole elongation and an increase in rosette diameter in 12 DAG seedlings, T3 p32449:CPD, p32449:GmCPD, and p32449:GmCPD2 transgenes did not. This is a consistent difference between the CPD and DWF4 phenotypes (Choe et al., 2001), showing that even though the two genes regulate adjacent steps in the brassinolide biosynthesis pathway, CPD and DWF4 transgenes have different effects on seedling growth and development.

Later in development, T3 p32449:GmCPD1 failed to establish an effect on rosette size 30 DAG or on seed yield at maturity in two transformation events (ME0819-1-6 and ME0819-3-3). This was also the case for the T3 p32449:GmCPD2 lines. These results were also at variance with previous findings with DWF4 transgenes. When 35S is used to express DWF4 in Arabidopsis (Choe et al., 2001) or p326 to express it in rice, shoot dry weight, seed number, and seed yield were enhanced.

A number of embodiments of the invention have been described. Nevertheless, it will be understood that various modifications may be made without departing from the spirit and scope of the invention. Accordingly, other embodiments are within the scope of the following claims.

What is claimed is:

1. A method of increasing the height of a plant, said method comprising:
a) introducing into a plurality of plant cells an exogenous nucleic acid comprising a polynucleotide sequence encoding a polypeptide having 90% or greater sequence identity to an amino acid sequence set forth in the Alignment Table, wherein said exogenous nucleic acid further comprises a broadly expressing promoter operably linked to said polynucleotide sequence; and
b) selecting a plant produced from said plurality of plant cells, wherein said plant has an increased height as compared to a corresponding control plant that does not comprise said exogenous nucleic acid.

2. A method of increasing the height of a plant, said method comprising:
a) introducing into a plurality of plant cells an exogenous nucleic acid comprising a polynucleotide sequence encoding a polypeptide having 90% or greater sequence identity to an amino acid sequence set forth in the Alignment Table, wherein said amino acid sequence is an amino acid sequence set forth in the Alignment Table other than the Arabidopsis amino acid sequence; and
b) selecting a plant produced from said plurality of plant cells, wherein said plant has an increased height as compared to a corresponding control plant that does not comprise said exogenous nucleic acid.

3. A method of increasing the height of a plant, said method comprising:
a) introducing into a plurality of plant cells an exogenous nucleic acid comprising a polynucleotide sequence encoding a polypeptide having 90% or greater sequence identity to an amino acid sequence set forth in the Alignment Table; and
b) selecting a plant produced from said plurality of plant cells, wherein said plant has an increased height as compared to a corresponding control plant that does not comprise said exogenous nucleic acid.

4. The method of claim 1, 2, or 3, wherein said exogenous nucleic acid comprises a polynucleotide sequence encoding a polypeptide having 95% or greater sequence identity to an amino acid sequence set forth in the Alignment Table.

5. The method of claim 1, 2, or 3, wherein said plant is a dicot.

6. The method of claim 1, 2, or 3, wherein said plant is a monocot.

7. The method of claim 1, wherein said broadly expressing promoter is selected from the group consisting of p326, p32449, p13679, YP0050, YP0144, and YP0190.

8. The method of claim 2 or 3, wherein said exogenous nucleic acid further comprises a promoter selected from the group consisting of a broadly expressing promoter, an inducible promoter, and a tissue-specific promoter operably linked to said polynucleotide.

9. The method of claim 1, 2, or 3, wherein the plant is selected from the group consisting of soybean, clover, alfalfa, and safflower.

10. The method of claim 1, 2, or 3, wherein the plant is selected from the group consisting of switchgrass, sudangrass, sorghum, corn, sugarcane, and oil palm.


SEQUENCE LISTING (35 sequences)

SEQ ID NO: 1 - DNA, 1682 bp, Arabidopsis thaliana
  misc_feature (1)..(1682): Ceres CLONE ID no. 36334
tccattaatactctctctccctcatcctctcttcttctctcatcatcatcttcttcttcaatggccttcaccgcttttctcctcctcctctcttccatcgccgccggcttcctcctcctactccgccgtacacgttaccgtcggatgggtctgcctccgggaagccttggtctccctctgataggagagacttttcagctgatcggagcttacaaaacagagaaccctgagcctttcatcgacgagagagtagcccggtacggttcggttttcatgacgcatctttttggtgaaccgacgattttctcagctgacccggaaacgaaccggtttgttcttcagaacgaagggaagctttttgagtgttcttatcctgcttccatttgtaaccttttggggaaacactctctgcttcttatgaaaggttctttgcataaacgtatgcactctctcaccatgagctttgctaattcttcaatcattaaagaccatctcatgcttgatattgaccggttagtccggtttaatcttgattcttggtcttctcgtgttctcctcatggaagaagccaaaaagataacgtttgagctaacggtgaagcagttgatgagctttgatccaggggaatggagtgagagtttaaggaaagagtatcttcttgtcatcgaaggcttcttctctcttcctctccctctcttctccaccacttaccgcaaagccatccaagcgcggaggaaggtggcggaggcgttgacggtggtggtgatgaaaaggagggaggaggaggaagaaggagcggagagaaagaaagatatgcttgcggcgttgcttgcggcggatgatggattttccgatgaagagattgttgacttcttggtggctttacttgtcgccggttatgaaacaacctccacgatcatgactctcgccgtcaaatttctcaccgagactcctttagctcttgctcaactcaaggaagagcatgaaaagattagggcaatgaagagtgattcgtatagtcttgaatggagtgattacaagtcaatgccattcacacaatgtgtggttaatgagacgctacgagtggctaacatcatcggcggtgttttcagacgtgcaatgacggatgttgagatcaaaggttataaaattccaaaagggtggaaagtattctcatcgtttagagcggttcatttagacccaaaccacttcaaagatgctcgcactttcaacccttggagatggcagagcaactcggtaacgacaggcccttctaatgtgttcacaccgtttggtggagggccaaggctatgtcccggttacgagctggctagggttgcactctctgttttccttcaccgcctagtgacaggcttcagttgggttcctgcagagcaagacaagctggttttctttccaactacaagaacgcagaaacggtacccgatcttcgtgaagcgccgtgattttgctacttgaagaagaagagacccatctgattttatttatagaacaacagtatttttcaggattaatttcttcttctttttttgcctccttgtgggtctagtgtttgacaataaaagttatcattactctataaagccttagcttctgtgtacataaaaaaaaaaaacttttgtttaccttatgcttgcataaatctcttctgcttcaatggt

SEQ ID NO: 2 - protein, 472 aa, Arabidopsis thaliana
  misc_feature (205)..(467): Pfam Name p450; Pfam Description Cytochrome P450
MAFTAFLLLLSSIAAGFLLLLRRTRYRRMGLPPGSLGLPLIGETFQLIGAYKTENPEPFIDERVARYGSVFMTHLFGEPTIFSADPETNRFVLQNEGKLFECSYPASICNLLGKHSLLLMKGSLHKRMHSLTMSFANSSIIKDHLMLDIDRLVRFNLDSWSSRVLLMEEAKKITFELTVKQLMSFDPGEWSESLRKEYLLVIEGFFSLPLPLFSTTYRKAIQARRKVAEALTVVVMKRREEEEEGAERKKDMLAALLAADDGFSDEEIVDFLVALLVAGYETTSTIMTLAVKFLTETPLALAQLKEEHEKIRAMKSDSYSLEWSDYKSMPFTQCVVNETLRVANIIGGVFRRAMTDVEIKGYKIPKGWKVFSSFRAVHLDPNHFKDARTFNPWRWQSNSVTTGPSNVFTPFGGGPRLCPGYELARVALSVFLHRLVTGFSWVPAEQDKLVFFPTTRTQKRYPIFVKRRDFAT

SEQ ID NO: 3 - DNA, 20 bp, Arabidopsis thaliana
  misc_feature (1)..(20): CPD_R2_primer
cagacccatccgacggtaac

SEQ ID NO: 4 - DNA, 20 bp, Arabidopsis thaliana
  misc_feature (1)..(20): CPD_F2_primer
cccttggagatggcagagca

SEQ ID NO: 5 - protein, 510 aa, Zea mays
  misc_feature (1)..(510): Ceres CLONE ID no. 339347
MDAGGTPPLLFLLAAAAALLGAALRWLLLAWRSAARTGRLPPGSTGLPLIGETLRLIAAYKTPNPEPFIDERVARHGSGVFTTHVFGERTVFSADPAFNRLLLAAEGRAVSCSYPSSIATLLGPRSLLLTAGPAHKRLHSLTLARLGRPASPPLLAHIDRLVLATLRDWGRPGAGAVVRLLDEAKKITFNLTVWQLVSIEPGPWTESLRREYVKLVDGFFSIPFPFAYLLPFTAYGQALKARKKVAGALREVIRKRMGEEAGTGPGAGRNGEKKDMVEELLEAEGGSFSVEEMVDFCLSLLVAGYETTSVLMTLAVKFLTETPTALAQLKEEHDSIRHRKGKDEQPLQWSDYKSMPFTQCVISETLRVANLISGVFRRANTDIHFKDYVIPKGCRIFASFRAVHLSPEHYENARAFDPWRWQQSKKEGVLVVGQDAQQGARASVFTPFGGGPRLCPGHELARVVVSVFLHRLVTRFSWEEAEEDRVVFFPTTRTLKGYPIILRRRPGWDF

SEQ ID NO: 6 - protein, 501 aa, Oryza sativa
  misc_feature (1)..(501): Rice_CPD_CYP90A3
MAAAALLLLAAAAAIVVVAMVLRWLLLLGGPAAGRLGKRALMPPGSTGLPLIGETLRLISAYKTPNPEPFIDERVARHGGVFTTHVFGERTVFSADPAFNRLLLAAEGRAVHSSYPSSIATLLGARSLLLTRGAAHKRLHSLTLTRLGRPASPPLLAHIDRLVLATMRQWEPAATVRLMDEAKKITFNLTVKQLVSIEPGPWTESLRREYVKLIDGFFSIPFPLANLLPFTTYGQALKARKKVAGALREVIKKRMEEKAENGGSIGDDEGKKEKKDMVEELLEAEGGSFSEEEMVDFCLSLLVAGYETTSMLMTLAVKFLTETPAALAELKEEHANIRDMKGKKQPLEWSDYKSMPFTQCVINETLRVGNIISGVFRRANTDIHYKDYTIPKGCKIFASFRAVHLNNEHYENARTFNPWRWQINNKLQNAVGANIFTPFGGGPRLCPGYELARVVVSIFLHHLVTRFSWEETEEDRLVFFPTTRTLKGYPINLRLLSESIC

SEQ ID NO: 7 - protein, 479 aa, Glycine max
  misc_feature (1)..(479): Ceres CLONE ID no. 690176
MASFIFTPVLFLLIISAVLLFLHRRSRCRRFRLPPGTLGLPFVGETLQLISAYKSDNPEPFMDQRVKRYGPIFTTHVFGEPTVFSTDPETNRFILLNEGKLFECSYPGSISNLLGKHSLLLMKGSLHKRMHSLTMSFANSSIIKDHLLVDIDRLIRLNLDSWSDRVLLMEEAKKITFELTVKQLMSFDPGEWTETLRKEYVLVIEGFFSVPLPLFSSTYRRAIKARTKVAEALTLVVRDRRKESVTEEKKNDMLGALLASGYHFSDEEIVDFMLALLVAGYETTSTIMTLAIKFLTETPLALAQLKEEHDQIRAKKSCPEAPLEWTDYKSMAFTQCVVNETLRVANIIGAIFRRAMTDINIKGYTIPKGWRVVASFRAVHLNPDHFKDARTFNPWRWQSNSEASSPGNVYTPFGGGPRLCPGYELARVVLSVFLHRIVTRYSWFPAEEDKLVFFPTTRTQKRYPIIVKRREESKLSKSP

SEQ ID NO: 8 - protein, 472 aa, Glycine max
  misc_feature (1)..(472): Ceres CLONE ID no. 574698
MASLPALPTLLLSFAAIFFTVLLLFLRRRQLRLPPGSYGLPLIGETLQLISAYKSDNPEPFIDERVERYGSIFTTHVFGEATVFSADPEVNRFILQNEGRLLDCSYPGSISNLLGKHSLLLMKGGLHKRMHSLTMSLANSSIIKDHLLHHIDRLVCLNLDAWSNRVFLMDQAKKITFELTVKQLMSFDPDEWTENLRKEYVLVIEGFFTLPFPLFSTTYRRAIKARTKVAEALTLVVRQRRKEYDEDKEKKNDMLGALLASGDHFSDEEIVDFLLALLVAGYETTSTIMTLAIKFLTETPLALAQLKEEHDQIRARSDPGTPLEWTDYKSMAFTQCVVNETLRVANIIGGIFRRARTDIDIKGYTIPKGWKVFASFRAVHLNPEHYKDARSFNPWRWQSNSSEATNPGNVYTPFGGGPRLCPGYKLARVVLSVFLHRIVTRFSWVPAEEDKLVFFPTTRTQKRYPIIVQRRD

SEQ ID NO: 9 - protein, 513 aa, Glycine max
  misc_feature (1)..(513): Public GI no. 19699122
MFETEHHTLLPLLLLPSLLSLLLFLILLKRRNRKTRFNLPPGKSGWPFLGETIGYLKPYTATTLGDFMQQHVSKYGKIYRSNLFGEPTIVSADAGLNRFILQNEGRLFECSYPRSIGGILGKWSMLVLVGDMHRDMRSISLNFLSHARLRTILLKDVERHTLFVLDSWQQNSIFSAQDEAKKFTFNLMAKHIMSMDPGEEETEQLKKEYVTFMKGVVSAPLNLPGTAYHKALQSRATILKFIERKMEERKLDIKEEDQEEEEVKTEDEAEMSKSDHVRKQRTDDDLLGWVLKHSNLSTEQILDLILSLLFAGHETSSVAIALAIFFLQACPKAVEELREEHLEIARAKKELGESELNWDDYKKMDFTQCVINETLRLGNVVRFLHRKALKDVRYKGYDIPSGWKVLPVISAVHLDNSRYDQPNLFNPWRWQQQNNGASSSGSGSFSTWGNNYMPFGGGPRLCAGSELAKLEMAVFIHHLVLKFNWELAEDDKPFAFPFVDFPNGLPIRVSRIL

SEQ ID NO: 10 - protein, 513 aa, Glycine max
  misc_feature (1)..(513): Public GI no. 2935342
MFETEHHTLLPLLLLPSLLSLLLFLILLKRRNRKTRFNLPPGKSGWPFLGETIGYLKPYTATTLGDFMQQHVSKYGKIYRSNLFGEPTIVSADAGLNRFILQNEGRLFECSYPRSIGGILGKWSMLVLVGDMHRDMRSISLNFLSHARLRTILLKDVERHTLFVLDSWQQNSIFSAQDEAKKFTFNLMAKHIMSMDPGEEETEQLKKEYVTFMKGVVSAPLNLPGTAYHKALQSRATILKFIERKMEERKLDIKEEDQEEEEVKTEDEAEMSKSDHVRKQRTDDDLLGWVLKHSNLSTEQILDLILSLLFAGHETSSVAIALAIFFLQACPKAVEELREEHLEIARAKKELGESELNWDDYKKMDFTQCVINETLRLGNVVRFLHRKALKDVRYKGYDIPSGWKVLPVISAVHLDNSRYDQPNLFNPWRWQQQNNGASSSGSGSFSTWGNNYMPFGGGPRLCAGSELAKLEMAVFIHHLVLKFNWELAEDDQPFAFPFVDFPNGLPIRVSRIL

SEQ ID NO: 11 - protein, 524 aa, Glycine max
  misc_feature (1)..(524): Public GI no. 13878393
MQPPASAGLFRSPENLPWPYNYMDYLVAGFLVLTAGILLRPWLWFRLRNSKTKDGDEEEDNEEKKKGMIPNGSLGWPVIGETLNFIACGYSSRPVTFMDKRKSLYGKVFKTNIIGTPIIISTDAEVNKVVLQNHGNTFVPAYPKSITELLGENSILSINGPHQKRLHTLIGAFLRSPHLKDRITRDIEASVVLTLASWAQLPLVHVQDEIKKMTFEILVKVLMSTSPGEDMNILKLEFEEFIKGLICIPIKFPGTRLYKSLKAKERLIKMVKKVVEERQVAMTTTSPANDVVDVLLRDGGDSEKQSQPSDFVSGKIVEMMIPGEETMPTAMTLAVKFLSDNPVALAKLVEENMEMKRRKLELGEEYKWTDYMSLSFTQNVINETLRMANIINGVWRKALKDVEIKGYLIPKGWCVLASFISVHMDEDIYDNPYQFDPWRWDRINGSANSSICFTPFGGGQRLCPGLELSKLEISIFLHHLVTRYSWTAEEDEIVSFPTVKMKRRLPIRVATVDDSASPISLEDH

SEQ ID NO: 12 - protein, 524 aa, Glycine max
  misc_feature (1)..(524): Public GI no. 18419825
MQPPASAGLFRSPENLPWPYNYMDYLVAGFLVLTAGILLRPWLWLRLRNSKTKDGDEEEDNEEKKKGMIPNGSLGWPVIGETLNFIACGYSSRPVTFMDKRKSLYGKVFKTNIIGTPIIISTDAEVNKVVLQNHGNTFVPAYPKSITELLGENSILSINGPHQKRLHTLIGAFLRSPHLKDRITRDIEASVVLTLASWAQLPLVHVQDEIKKMTFEILVKVLMSTSPGEDMNILKLEFEEFIKGLICIPIKFPGTRLYKSLKAKERLIKMVKKVVEERQVAMTTTSPANDVVDVLLRDGGDSEKQSQPSDFVSGKIVEMMIPGEETMPTAMTLAVKFLSDNPVALAKLVEENMEMKRRKLELGEEYKWTDYMSLSFTQNVINETLRMANIINGVWRKALKDVEIKGYLIPKGWCVLASFISVHMDEDIYDNPYQFDPWRWDRINGSANSSICFTPFGGGQRLCPGLELSKLEISIFLHHLVTRYSWTAEEDEIVSFPTVKMKRRLPIRVATVDDSASPISLEDH

SEQ ID NO: 13 - protein, 457 aa, Glycine max
  misc_feature (1)..(457): Public GI no. 4006922
MIPNGSLGWPVIGETLNFIACGYSSRPVTFMDKRKSLYGKVFKTNIIGTPIIISTDAEVNKVVLQNHGNTFVPAYPKSITELLGENSILSINGPHQKRLHTLIGAFLRSPHLKDRITRDIEASVVLTLASWAQLPLVHVQDEIKKMTFEILVKVLMSTSPGEDMNILKLEFEEFIKGLICIPIKFPGTRLYKSLKAKERLIKMVKKVVEERQVAMTTTSPANDVVDVLLRDGGDSEKQSQPSDFVSGKIVEMMIPGEETMPTAMTLAVKFLSDNPVALAKLVEENMEMKRRKLELGEEYKWTDYMSLSFTQNVINETLRMANIINGVWRKALKDVEIKGYLIPKGWCVLASFISVHMDEDIYDNPYQFDPWRWDRINGSANSSICFTPFGGGQRLCPGLELSKLEISIFLHHLVTRYSWTAEEDEIVSFPTVKMKRRLPIRVATVDDSASPISLEDH

SEQ ID NO: 14 - protein, 483 aa, Glycine max
  misc_feature (1)..(483): Public GI no. 45260636
MDFIIYLFLSFSISLITFLLLRAAAAAHFRRRKTRLPPGTLGLPFIGETLQLISAYKTENPEPFIDDRVSKYGNIFTTHIFGEPTVFSTDAETNRFILQNEGRPFESSYPSSLQNLLGKHSLLLMRGSLHKRMHSLTMSFANSSILKDHLLADIDRLVRLNLDSWTGRVFLMEEAKKITFNLTVKQLMSLDPCEWTEKLMKEYMLVIEGFFTIPLPFFSSTYRKAIQARRKVAEALGLVVKERRKERGGGERLKNDMLEALFEGDGVEGFSDEVIVDFMLALLVAGYETTSTIMTLAVKFLTETPHALSLLKEEHEEIRLRKGDVESLLWEDYKSMPFTQCVVNETLRVGNIISGVFRRTMTDINIKGYTIPKGWKVFACFRAVHLDHEHFKDARTFDPWRWQSNAGSTSSPNVFTPFGGGPRRCPGYELARVELSVFLHHLVTRHSWVPAEPDKLVFFPTTRMQKRYPIIVQRRSLFDPCKE

SEQ ID NO: 15 - protein, 501 aa, Glycine max
  misc_feature (1)..(501): Public GI no. 60677685
MAAAALLLLAAAAAAVVVAMALRWLLLLGGPAAGRLGKRARMPPGSTGLPLIGETLRLISAYKTPNPEPFIDERVARHGGVFTTHVFGERTVFSADPAFNRLLLAAEGRAVHSSYPSSIATLLGARSLLLTRGAAHKRLHSLTLTRLGRPASPPLLAHIDRLVLATMRQWEPAATVRLMDEAKKITFNLTVKQLVSIEPGPWTESLRREYVKLIDGFFSIPFPLAYFLPFTTYGQALKARKKVAGALREVIKKRMEEKAENGGSIGDDEGKKEKKDMVEELLQAEGGSFSEEEMVDFCLSLLVAGYETTSVLMTLAVKFLTETPAALAELKEEHANIRDMKGKNQPLEWSDYKSMPFTQCVINETLRVGNIISGVFRRANTDIHYKDYTIPKGCKIFASFRAVHLNNEHYENARTFNPWRWQINNKLQNAVGANIFTPFGGGPRLCPGYELARVVVSIFLHHLVTRFSWEETEEDRLVFFPTTRTLKGYPINLRLLSESIC

SEQ ID NO: 16 - protein, 501 aa, Glycine max
  misc_feature (1)..(501): Public GI no. 60677683
MAAAALLLLAAAAAIVVVAMVLRWLLLLGGPAAGRLGKRALMPPGSTGLPLIGETLRLISAYKTPNPEPFIDERVARHGGVFTTHVFGERTVFSADPAFNRLLLAAEGRAVHSSYPSSIATLLGARSLLLTRGAAHKRLHSLTFTRLGRPASPPLLAHIDRLVLATMRQWEPAATVRLMDEAKKITFNLTVKQLVSIEPGPWTESLRREYVKLIDGFFSIPFPLANLLPFTTYGQALKARKKVAGALREVIKKRMEEKAENGGSIGDDEGKKEKKDMVEELLEAEGGSFSEEEMVDFCLSLLVAGYETTSMLMTLAVKFLTETPAALAELKEEHANIRDMKGKKQPLEWSDYKSMPFTQCVINETLRVGNIISGVFRRANTDIHYKDYTIPKGCKIFASFRAVHLNNEHYENARTFNPWRWQINNKLQNAVGANIFTPFGGGPRLCPGYELARVVVSIFLHHLVTRFSWEETEEDRLVFFPTTRTLKGYPINLRLLSESIC

SEQ ID NO: 17 - protein, 502 aa, Glycine max
  misc_feature (1)..(502): Public GI no. 34902330
MASITSELLFFLPFILLALLTFYTTTVAKCHGGHWWRGGTTPAKRKRMNLPPGAAGWPLVGETFGYLRAHPATSVGRFMEQHIARYGKIYRSSLFGERTVVSADAGLNRYILQNEGRLFECSYPRSIGGILGKWSMLVLVGDPHREMRAISLNFLSSVRLRAVLLPEVERHTLLVLRAWPPSSTFSAQHQAKKFTFNLMAKNIMSMDPGEEETERLRREYITFMKGVVSAPLNLPGTPYWKALKSRAAILGVIERKMEERVEKLSKEDASVEQDDLLGWALKQSNLSKEQILDLLLSLLFAGHETSSMALALAIFFLEGCPKAVQELREEHLGIARRQRLRGECKLSWEDYKEMVFTQCVINETLRLGNVVRFLHRKVIKDVHYKGYDIPSGWKILPVLAAVHLDSSLYEDPQRFNPWRWKSSGSSGGLAQSSSFMPYGGGTRLCAGSELAKLEMAVFLHHLVLNFRWELAEPDQAFVFPFVDFPKGLPIRVHRIAQDDEQE

SEQ ID NO: 18 - protein, 474 aa, Glycine max
  misc_feature (1)..(474): Public GI no. 9587211
MVSLPTLLLLFAASAAAIFLHRAFSRRKFRLPPGSYGLPFIGETLQLISAYKSSNPEPFMDERVRRYGSIFMTHVFGEPTVFSADPELNRFILQNEGKLLDCSYPGSISNLLGKHSLLLMKGALHKRMHSLTMSFANSSIIKDHLLHHIDRLIGLNLDTWSDRVTLMDQAKKITFELTVKQLMSFDPDEWTESLRKEYVLVIEGFFTLPLPLFSTTYRRAIKARTKVAEALTLVVRQRREEYNQGKEKKSDMLGALLASGDHFSDDQIVDFLLALLVAGYETTSTIMTLAVKFLTETPLALAQLKEEHDQIRARSDPGAPLEWTDYKSMVFTQHVVNETLRVANIIGGIFRRATTDIDIKGYTIPKGWKVFASFRAVHLNPEYYKDARTFNPWRWQSNSSEAANPANVYTPFGGGPRLCPGYELARVVLSVFLHRIVTRFSWVPAEEDKLVFFPTTRTQKRYPIIVKRRNANHV

SEQ ID NO: 19 - DNA, 2003 bp, Arabidopsis thaliana
  misc_feature (1)..(2003): Ceres PROMOTER ID no. 32449
ttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataa

SEQ ID NO: 20 - DNA, 1019 bp, Arabidopsis thaliana
agtcgattggaaacgttgcaagattattgattgtgagaagagtgctcaaggtagtactgatttctgtaaagctcacggtggtgggaaacgatgttcttggggagatgggaaatgtgagaaatttgctagaggaaagagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaaccattgcact

SEQ ID NO: 21 - DNA, 1003 bp, Arabidopsis thaliana
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 22 - DNA, 1144 bp, Arabidopsis thaliana
agtcgattgggattgttggggcatgtgtgatgcgtttaacgattctaacagtatatgaaattatattttttggtcttgttatttgtctaaaaacctatatttttctcgtaagaatattgtaagagttatttttcgaaaatttaaataatgattcgatcaacactttttctcattttatcaaacccctttgattgaatagaccgctaaaacaatttgcttgattggtctttcttacaacgactaagttacaaatgtgactgaaagttaccgatcaaacccatgaaaaaaacttgagcccatataccttgctatggatttggcacacagaccaagctttcgaagcaactgtttggttgattcggaattgttttctgataataaataatatttatattattcgttatgtgtttgtgataggataactcggaacataagcaactttaacttgtggcgatgcgagaaccaatgtgaaataggcatgtgagagaccacattgtcccacagcttttgtcctcttcacccccgcaattatattaccattaattaatcacatagttatcgttttccaaatcgtaatatacatatcgtagttgttcatctttaatctattttcggtaatctaacaaaaagaaagatatctcgtagtgaaaatacgaatatcagtgctttttatgcaacaattatgacattaggtatcgttactcaaagttaaatgaatacaatctagacgacgcttaaaaaacgaatagatgatggaatcacgacttaacactagaattaccatggaatataggcaatttgcgaatttattcaaccaaaccaaaaatcgacagtgttatttagtcaaaccttctaagaaaaagtgacccatttccaaggaacgatgaataaaaaaaccggaccaatgttgttccgacataagtcactagtggcaaagtcataatttagacaaaggaaaggggcctttcttgcacaattttgcatataagagctctctctcctcctcgttccattgcactggtctattccactcccactaaacattccttctctcgctcactcttctccaatccttattttattttttgaaagtttaaaattttatacaacatatcaatttggggtagaaaaattcgaaagaaaa

SEQ ID NO: 23 - DNA, 1002 bp, Arabidopsis thaliana
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 24 - DNA, 1514 bp, Arabidopsis thaliana
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 25 - DNA, 999 bp, Arabidopsis thaliana
tacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccataaaacatcaccacctctgttgtcatctcatgaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaacc

SEQ ID NO: 26 - DNA, 458 bp, Arabidopsis thaliana
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaaccc

SEQ ID NO: 27 - DNA, 1954 bp, Arabidopsis thaliana
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 28 - DNA, 20 bp, Artificial Sequence
  Synthetically generated oligonucleotide
cgagggtttctctcttcctc

SEQ ID NO: 29 - DNA, 20 bp, Artificial Sequence
  Synthetically generated oligonucleotide
tcttacaatttcccgctctg

SEQ ID NO: 30 - DNA, 20 bp, Artificial Sequence
  Synthetically generated oligonucleotide
atggcatctttcatcttcac

SEQ ID NO: 31 - DNA, 21 bp, Artificial Sequence
  Synthetically generated oligonucleotide
ccttattcgtcttcttcgttc

SEQ ID NO: 32 - DNA, 21 bp, Artificial Sequence
  Synthetically generated oligonucleotide
tcattaaagcaggactctagc

SEQ ID NO: 33 - DNA, 20 bp, Artificial Sequence
  Synthetically generated oligonucleotide
ctacgtcagagagtgcattc

SEQ ID NO: 34 - DNA, 21 bp, Artificial Sequence
  Synthetically generated oligonucleotide
gggatccaaagtctttgcatc

SEQ ID NO: 35 - DNA, 20 bp, Artificial Sequence
  Synthetically generated oligonucleotide
ttgtaagctgatatgagctg